(19) United States
(12) Patent Application Publication
(10) Pub. No.: US 2003/0120202 A1
(43) Pub. Date: Jun. 26, 2003

(54) Magnetic extracorporeal circuit for removal of medical agents

(76) Inventor: Lucas S. Gordon, Sammamish, WA (US)

Correspondence Address: Ronald M. Anderson, LAW OFFICES OF RONALD M. ANDERSON, Suite 507, 600 - 108th Avenue N.E., Bellevue, WA, 98004, US

(21) Appl. No.: 10/032,211

(22) Filed: Dec. 21, 2001

(51) Int. Cl. A61M 1/00

(52) U.S. Cl. 604/28; 600/12; 604/500

(57) ABSTRACT

A method and system for delivering a medical agent to targeted tissues in a patient, and subsequently removing systemically distributed medical agent to minimize exposure of non-targeted portions of a patient's body to the medical agent. The patient is treated with a targeted medical agent that includes a therapeutic component, a targeting component, and a magnetically attracted component. The targeting component, such as an antibody known to be attracted to an antigen present at the target, causes a concentration of the targeted medical agent to occur at the targeted tissue. The medical agent not concentrated at the targeted tissue is removed by withdrawing and magnetically filtering a portion of the patient's blood to remove the targeted medical agent, and returning the filtered blood to the patient.

### FIELD OF THE INVENTION

The present invention is generally directed to a method and a system for removing a medical agent circulating in a bodily passage, and more specifically, is directed to the delivery of a therapeutic or diagnostic agent to a localized site in a patient and the removal of unnecessary or excess amounts of such an agent from a patient, to prevent an undesirable accumulation of the agent in a patient's body.

### BACKGROUND OF THE INVENTION

Delivery of pharmacological and diagnostic agents to specific targeted tissue areas, with minimum systemic distribution and uptake by other tissues, is a continuing goal of drug delivery systems. Unfortunately, many powerful drugs that are thus delivered into targeted tissue areas can produce undesirable toxic effects in other areas of the body.

For instance, it is known that appropriately sized liposomes accumulate preferentially in tumoral tissues. This accumulation is due to the high permeability of capillaries and venules found in tumoral tissues. Particles as large as 400 nanometers in diameter can pass through the spaces between the endothelial cells lining the micro-vasculature of cancerous tumors. By introducing chemotherapy agents into liposome carriers, such agents can be preferentially delivered into the tumor, with minimal uptake of the chemotherapy agents into normal tissue, whose endothelium generally prevents the passage of such carriers. This type of preferential distribution is termed “passive targeting,” since the liposomes accumulate within any tissue having porous vasculature. After intravascular administration and circulation of the chemotherapeutic liposomes, the reticuloendothelial system (RES) gradually removes the carriers and they accumulate in the liver and spleen, and are eventually eliminated from the body.

In order to extend the circulating time within the vasculature, thereby increasing the loading of the tumor with liposomes, various surface treatments may be used to reduce the clearance of the liposomes from a patient's body by the RES. One such surface treatment adds polyethylene glycol (PEG) molecules to the surface of the liposome. Currently several PEG/liposome/chemotherapy carriers are in use, such as Caelyx™ liposomally encapsulated doxorubicin. However, even with preferential uptake of the carriers by passive targeting, it is estimated that less than five percent of the total administered chemotherapy agent actually reaches the tumor. The rest of the chemotherapy agent is distributed throughout the body to non-targeted tissues, resulting in significant, often dose limiting, toxic side effects.

Active targeting of tissues with a medicinal agent increases the effectiveness of the delivery system. The goal of active targeting is to achieve one or more of the following effects: (1) increase the rate at which the medical agent is concentrated in the target tissue; (2) increase retention of the medical agent, once it is in the target tissue; and, (3) increase the penetration of the medical agent into the target tissue. A common method to achieve one or more of these goals is to attach a targeting moiety, such as an anti-tumor monoclonal antibody, to the medical agent (for example, an anti-tumor agent encapsulated in a liposome). The antibody only recognizes a specific antigen on the surface of a cancer cell, and attaches to a cancer cell when one is encountered, so that the antibody is both attracted to and retained by the tumor. Some antibodies actually facilitate the penetration of a carrier into a cancer cell. However, even “active targeting” results in less than 10 percent of the administered medical agent load reaching the tumorous tissues.

Many medical agents, including gene therapy agents, antibiotic agents, and radiation therapy agents, have beneficial applications when used in a specific location within the body, yet are harmful if distributed systemically. In addition to therapeutic and diagnostic agents that are intentionally administered into a patient's vasculature, many other undesirable agents such as bacteria, virus, and prions, occur naturally, or as a result of disease condition. Other undesirable agents include cancer cells and certain proteins, enzymes, antibodies, antigens, and the like.

Many systems for separating undesirable agents from blood are known. U.S. Pat. No. 3,959,128 (Harris) describes a process for removing endotoxins from blood by intimately contacting blood contaminated with endotoxin with a non-polar aliphatic synthetic polymer capable of adsorbing that endotoxin. The method provides for removing a quantity of blood from the patient (an animal, as disclosed by Harris), passing the blood through a column containing the polymer, and then reinfusing the blood back into the patient. Such affinity type separators require that each molecule of the endotoxin be brought into intimate contact with the surface of the polymer in the separation device. This step is very difficult to carry out, especially when the fluid contains a large quantity of cells, as does blood. Affinity type systems generally require very low blood flows, special mixing devices, and large surface areas to be reasonably effective. Unfortunately, the mixing devices and large surface areas employed can produce damaging, hemolytic effects on the sensitive cells within the blood.

U.S. Pat. No. 4,820,261 (Schmoll et al.) discloses another system for removing a substance from blood. A catheter is utilized for withdrawing blood from a vein that drains an area in a person's body containing a tumor. The withdrawn blood is pumped through a container having immobilized antibodies, which remove the substance. The blood is then returned to the patient.

In a related device described in U.S. Pat. No. 4,464,165 (Pollard, Jr.), whole blood is treated to remove immunoglobulins (IgGs) and immune complexes, by contacting the blood with an immunoadsorbent material. The immunoadsorbent material described is a deactivated protein A bearing Staphylococcus aureus bacteria that is immobilized in a polymeric matrix. The matrix material comprises gel-like beads having an average diameter ranging from 100 to 1000 microns.

U.S. Pat. No. 4,261,828 (Brunner et al.) discloses apparatus for the detoxification of blood, using an extracorporeal container having an inlet opening and an outlet opening. Within the container are a plurality of enzyme carriers, including a blood compatible embedding material that is impermeable with respect to corpuscular blood components, but permeable with respect to substances in solution, thus providing a molecular sieve.

In U.S. Pat. No. 4,816,409 (Tanaka et al.), another extracorporeal blood treatment device is disclosed, which eliminates tumor cells from blood by bringing the blood into contact with a water-insoluble anti-tumor monoclonal antibody.

U.S. Pat. No. 6,251,394 (Nilsson et al.) describes a method and system for enhanced in vivo clearance of diagnostic and/or therapeutic agents by extracorporeal depletion. Nilsson et al. disclose the use of an agent, such as tumor-selective monoclonal antibodies labeled with a gamma-emitting radionuclide, which is conjugated with biotin. The immunoconjugate is injected into a patient and is selectively concentrated within tumors. After a period of time sufficient to enable adequate uptake, the excess immunoconjugate is removed by extracorporeal immunoadsorption of plasma through an avidin column. Avidin is known to strongly bind to biotin. Thus, the biotin conjugate is removed from plasma that is drawn from the patient and passed through an avidin adsorbent column. The immunoconjugate depleted plasma is remixed with blood and returned to the patient. While effective for removal of biotin bound agents, the process is slow, and cannot be used for non-biotin bound medical agents.

While many attempts have been made to deliver medical agents to specific areas of the body, relatively few attempts have been made to prevent subsequent systemic distribution of the agents. One such attempt is implemented using an apparatus described by Boddie, in U.S. Pat. No. 4,192,302. The apparatus described therein is an extracorporeal hepatic isolation and perfusion circuit. Blood is withdrawn from the portal vein and passed through an oxygenator, where it is oxygenated and receives chemotherapy agents. The oxygenated blood/chemotherapy agents are then returned to the hepatic artery. While functional for its intended use, the apparatus is quite complex and expensive. A simpler system was described by Bodden in U.S. Pat. No. 5,069,662. Again, an extracorporeal circuit is employed; the circuit isolates and removes venous blood from the liver. The blood is pumped through a device to remove a portion of a chemotherapeutic agent by hemofiltration with a microporous filter, and the treated blood is returned to the patient's venous system.

In an attempt to localize delivery of a therapeutic agent, Freeman et al. proposed that magnetically directed iron particles might be used to carry chemicals to particular areas of the body (J. App. Phys., Supp. Vol. 31, 404S-405S, May 1960). As disclosed, a magnetic field is placed near the desired area of the body in order to retain the particles that are being carried through the area in the blood stream. Myers et al. also suggested a similar system utilizing carbonyl iron particles as carriers of therapeutic agents for site specific delivery (Amer. J. Roentg., 90, 1068-1077 November 1963). In neither case, when the magnetic field is removed, are the particles free to move with the flow of blood away from the target site. Any residual medical agent still bound to the particles would become essentially systemic. In either article are methods or systems described or suggested about how to prevent the systemic delivery of residual drugs disposed on the iron particles.

U.S. Pat. No. 4,345,588 (Widder et al.) describes magnetically localized biodegradable microspheres containing a therapeutic agent. The microspheres are intravascularly administered and magnetically localized in a target capillary bed to concentrate the effect of the agent in the target capillary bed. The microspheres described by Widder have a preferred size of 0.5 to 1.5 microns. This size is preferred to prevent occlusion of the capillary system by the particles and resulting ischemia of the surrounding tissue. The microspheres containing the therapeutic agent are released into the general circulatory system when the magnetic force is removed and are thus free to induce adverse systemic effects to non target areas.

In U.S. Pat. No. 5,123,901 (Carew), an extracorporeal method for removing undesirable pathogenic or toxic agents from a body fluid is described. The method requires the introduction of paramagnetic beads into blood withdrawn from the patient. The beads have a coating of antibodies that bind to the pathogen or toxin that is to be removed (such as cells or viruses). The withdrawn blood is then directed to a magnetic filtering device that captures the paramagnetic beads, and the purified blood is returned to the patient.

A similar method, described in U.S. Pat. No. 5,980,479 (Kutushov), also uses an extracorporeal circuit and magneto-conductive particles capable of adsorbing toxins. First, blood is withdrawn and mixed with the magneto-conductive particles. Then the blood containing the magneto-conductive particles is passed through a magnetic field region of 30-100 mT, where the particles and adsorbed toxins are removed. The particle free blood is then returned to the patient.

Based upon the preceding discussion, it would clearly be desirable to provide a simple and effective method to extracorporeally removing excess, systemically circulating medical agents, which have been administered to a patient. The prior art does not disclose a method that enables medical agents to readily be removed outside the body of a patient in an efficient manner.

### SUMMARY OF THE INVENTION

The present invention relates to methods for delivering a medical agent to tissues in a patient's body and subsequently removing a portion of the medical agent from a bodily fluid. Specifically, a targeted medical agent that includes a magnetically sensitive component is administered to a patient. A portion of the targeted medical agent accumulates at a target location, and another portion of the targeted medical agent is systemically distributed throughout the body of the patient. The portion that is systemically distributed may have undesirable effects on the patient, but is removed by extracorporeally magnetically filtering the patient's bodily fluids.

In one embodiment, blood is withdrawn from the patient and is passed through a magnetic filter to remove at least a portion of the targeted medical agent from the withdrawn blood. The filtered blood is then returned to the patient, and the process is repeated until the amount of systemically distributed targeted medical agent remaining is reduced to at least a desired level. Bodily fluids other than blood, such as lymph, bile, and plasma, can also be filtered using this method.

In one embodiment, the extracorporeal magnetic filtering is a continuous process. A small portion of a patient's bodily fluids is removed, filtered, and returned in a closed loop process that continues until a desired systemic reduction of the targeted medical agent is achieved. Preferably in such a process, the bodily fluids are removed and returned at different points on the body of the patient. A pump or arterial pressure is employed to drive the bodily fluids through the magnetic filter.

In another embodiment, a volume of bodily fluid is withdrawn, filtered, and returned in a batch process. In this batch process, filtered bodily fluids are temporarily held in a reservoir. Once a desired volume of bodily fluid has been filtered, the contents of the reservoir are returned to the patient's body. Note that the batch process can be implemented by withdrawing and returning the bodily fluid to the same location in the patient's body. Preferably a pump first pumps a desired volume of bodily fluid through the magnetic filter and into the reservoir, then the pump is reversed and the filtered bodily fluid once again passes though the magnetic filter and returns to the patient. The volume processed per batch can be controlled by the number of pump cycles. If a molecular sieve or other type of sieve filter component were employed, passing the filtered bodily fluid back through the sieve filter would back flush the filter and tend to reintroduce the filtered component back into the fluid. However, reversing directions in a magnetic filter does not contaminate the filtered fluid. In fact, more magnetically sensitive materials should be removed as the filtered bodily fluids are passed back through the magnetic filter in the opposite direction.

Because the magnetically sensitive materials incorporated into a targeted medical agent are likely to be only weakly sensitive to magnetic fields, a preferred method employs a magnetic filter having a strong magnetic field, and the bodily fluids will pass through the filter at relatively low flow rates, thereby providing long residence times. Preferred parameters include flow rates of about 200 milliliters per minute, residence times of about five seconds, and magnetic fields of at least about 0.1 Tesla.

The targeted medical agent preferably includes a therapeutic component, a targeting component, and a magnetically sensitive material. The targeting component can be passive, such as an appropriately sized liposome or polymer sphere, or active, such as an antibody. In at least one embodiment, the magnetically sensitive material can be used in conjunction with a magnetic field directed to the target location for active targeting of a desired treatment site with the therapeutic component.

A still further aspect of the present invention relates to apparatus for magnetically filtering medical agents from a bodily fluid withdrawn from a patient. The functions carried out by elements of the apparatus are generally consistent with the steps of the method described above.

### BRIEF DESCRIPTION OF THE DRAWING FIGURES

The foregoing aspects and many of the attendant advantages of this invention will become more readily appreciated as the same becomes better understood by reference to the following detailed description, when taken in conjunction with the accompanying drawings, wherein:

FIG. 1 is a flow chart diagram illustrating the logical steps implemented to reduce the amount of a medical agent systemically distributed throughout the body of a patient, in accord with the present invention;

FIG. 2 is a schematic illustration of the principal components of an extracorporeal circuit for removing medical agents that include a magnetically sensitive component in accord with the present invention;

FIG. 3 is a schematic view of a medical agent that incorporates a magnetically sensitive component;

FIG. 4 is a cross-sectional view of a magnetic filter including a magnetic separation chamber and a magnet assembly, suitable for use in the circuit shown in FIG. 2;

FIG. 5 is a cross-sectional view of the magnetic filter of FIG. 4, taken along section line 5-5;

FIG. 6 is a cross-sectional view of another embodiment of a magnetic filter in which the magnet assembly is disposed inside the magnetic separation chamber, which is also suitable for use in the circuit shown in FIG. 2;

FIG. 7 is a cross-sectional view of the magnetic filter shown in FIG. 6, taken along section line 7-7;

FIG. 8 is a schematic illustration of another embodiment of an extracorporeal circuit for removing medical agents with magnetically sensitive components; and

FIG. 9 is a schematic illustration of another medical agent that includes a magnetically sensitive component, as well as an antibody targeting component.

### DESCRIPTION OF THE PREFERRED EMBODIMENT

In the present invention, a targeted medical agent is delivered to tissue in a patient's body, and subsequently, unnecessary medical agent is removed from the patient's body. The targeted medical agent includes a magnetically sensitive component and a targeting component. Some of the medical agent is concentrated at targeted tissue, and the medical agent not concentrated at the targeted location is removed by withdrawing and magnetically filtering a portion of the patient's bodily fluid to remove at least some of the medical agent/magnetically sensitive construct. The filtered bodily fluid is returned to the patient.

Before describing the invention in greater detail, it will be helpful to define several terms. The definitions provided below should be consistently applied to these terms both as they are used in this disclosure, and in the claims that follow.

An “extracorporeal circuit” refers to any device employed for externally processing or modifying bodily fluids, such as blood. Such bodily fluids may be withdrawn from a duct, blood vessel, body cavity, or hollow body organ. Extracorporeal circuits, without limitations, include continuous fluid flow systems, batch systems, single needle access systems, double needle access systems, and other systems known in the art for accessing and conveying fluid flowing in body passages.

A “carrier” refers to a construct or material used to transport a medical agent to tissue within a patient's body. Examples, without limitation, include organic particles, inorganic particles, liposomes, niosomes, proteins, lipids, polymers, peptides, lipopolymers, gas bubbles, biological cells such as virus or bacteria, prions, antibodies, antigens, hydrogels, polymers, dendrimers, and the like.

A “medical agent” encompasses therapeutic agents, diagnostic agents, imaging agents, cellular agents, and other agents that are of medicinal value. A medical agent may also comprise conjugated agents, incorporating functional components including carriers (such as microparticles) or encapsulating agents (such as liposomes), therapeutic agents (such as drugs or pro-drugs), imaging agents (such as radioactive components), and targeting agents (such as antibodies). Exemplary examples of carriers and encapsulants include, without limiting the present invention to use with such examples, polymer solids, polymer gels, niosomes, and microscopic bubbles.

A “therapeutic agent” refers to any drug, chemical, or other material that is used in the treatment of a disease or disorder. Examples, without limitation, include gene therapy agents, antibiotics, chemotherapy agents, anti-neoplastics, hormones, antivirals, radiation (via radiation sources such as cobalt, radium, radioactive sodium iodide, etc.), anticoagulants, enzymes, hepatoprotectants, vasodilators, prodrugs, and the like. A therapeutic agent may also combined with another liquid such as physiologic saline or the like. Any therapeutic agent that can be adhered to the surface of a carrier, impregnated into a carrier, or into a second material that is itself adhered to the surface of a carrier, may be administered using the devices and methods herein.

A “diagnostic agent” refers to any chemical or other material that is used to determine the nature of a disease or disorder. Exemplary examples of diagnostic agents include, without limiting the present invention to use with such examples, dyes that react with metabolic products of a particular disease, and radioactive materials that bind to and thereby indicate the presence of disease-causing entities within a patient's body. As is the case with therapeutic agents, any diagnostic agent that can be adhered to the surface of a carrier, impregnated into a carrier, or into a second material that is itself adhered to the surface of a carrier, may be administered using the devices and methods described herein.

An “imaging agent” refers to any material comprising an agent that aids the use of various types of body scanners to distinguish tissue from surrounding tissues more readily. Examples, without limitation, include radiopaque contrast agents imaged by x-ray systems, ferromagnetic or superparamagnetic metal particles imaged by magnetic resonance, and gas bubbles, low density spheres and hollow spheres imaged by ultrasound, radionuclides (such as In-111, Tc-99, I-121, I-123, I-125 F-18, Y-90, Ga-67, Ga-68) imaged by imaging systems sensitive to radiation, contrast agents such as chelated diethylenetriamine pentaacetic acid manganese for magnetic resonance imaging (MRI), and unpair spin atoms and free radicals (such as Fe, lanthides and Gd) for positron emission tomography (PET) and single photon emission tomography (SPET).

The term “magnetically sensitive” refers to a device or material that can be immobilized or manipulated by a magnetic field. Exemplary materials, without limiting the invention, include ferromagnetic, paramagnetic, and superparamagnetic materials.

The term “treating” refers to, without limitation, any act of monitoring, diagnosing, curing, or maintaining the status of a patient using a medical agent.

The term “passive targeting” refers to a targeting paradigm in which a characteristic of a targeting component causes that targeting component to naturally be concentrated at a target tissue location. As noted in the Background of the Invention, one example of passive targeting is the use of appropriately sized liposomes or microparticles that have the ability to pass through capillary vessels and thus tend to accumulate at locations adjacent to such capillary vessels. For example, PEG coated liposome complexes preferentially accumulate in tumoral tissue due to the large gaps between endothelial cells in the microcirculation of the tumors. The rate of localization in the tumoral tissue is dependent on several factors, including the concentration of targeted medical agents in the blood, the level of interstitial pressure within the tumor, and the size and surface characteristics of the targeted medical agent. Pegylated liposomes of approximately 100 nanometers in size will significantly localize in a tumor after about four or more hours of vascular circulation. Typically, such liposome-based targeted medical agents incorporate a chemotherapy agent (such as doxorubicin) as a therapeutic component, and are administered intravenously over a period of an hour or more.

In contrast, exemplary active targeting examples include magnetic targeting and antibody targeting. Magnetic targeting employs a magnetically sensitive material added to a medical agent, and the resulting targeted medical agent is introduced into a patient. A magnetic field is focused at the target location, and the magnetically targeted medical agent is concentrated at the target location by the applied magnetic field. In antibody targeting, a specific antibody is added to a medical agent. The specific antibody selected is naturally attracted to corresponding antigens that are present in the target tissue to a greater extent than in other types of tissue. As an antibody targeted medical agent is circulated throughout a patient's body by natural circulatory processes, the antibody targeted medical agent tends to accumulate at the target tissue.

It should be noted that many different types of molecules have been shown to preferentially target specific types of tissues or cells in the body, and can similarly be used for active targeting. Carbohydrate polymers may be used as targeting agents for drug delivery carriers. For example, one such polymer is hyaluronan. Breast cancer cells often contain a molecule called CD-44 that binds to hyaluronan (also known as hyaluronic acid), which is found in many types of sugar molecules. Therefore an active targeting system for breast cancer may consist of attaching sugar molecules containing hyaluronan to a carrier such as a liposome.

Other agents having an affinity for certain types of tissue, and thus can be used for active targeting, include: (1) natural fatty acids (such as docosahexaenoic acid) which have been shown to have enhanced uptake into tumors; (2) combretastatins (small organic molecules found in the bark of the African Bush Willow), which have been shown to target certain types of tumors; (3) stem cells, which have been reported to express some passive targeting abilities; (4) proteins (such as a-Fetoprotein and Transferrin), which can passively target specific tissues; and (5) peptides—any member of a class of compounds of low molecular weight that yield two or more amino acids on hydrolysis, and that form the constituent parts of proteins—(such as melanocortins and cyclic decapeptides) which can be used to target a variety of tissues or specific types of cells including some types of tumor cells.

The term “about” means that the characteristic modified by such term may vary by 0-20% from the norm for that characteristic, and still be within the scope of this invention, unless expressly stated to the contrary.

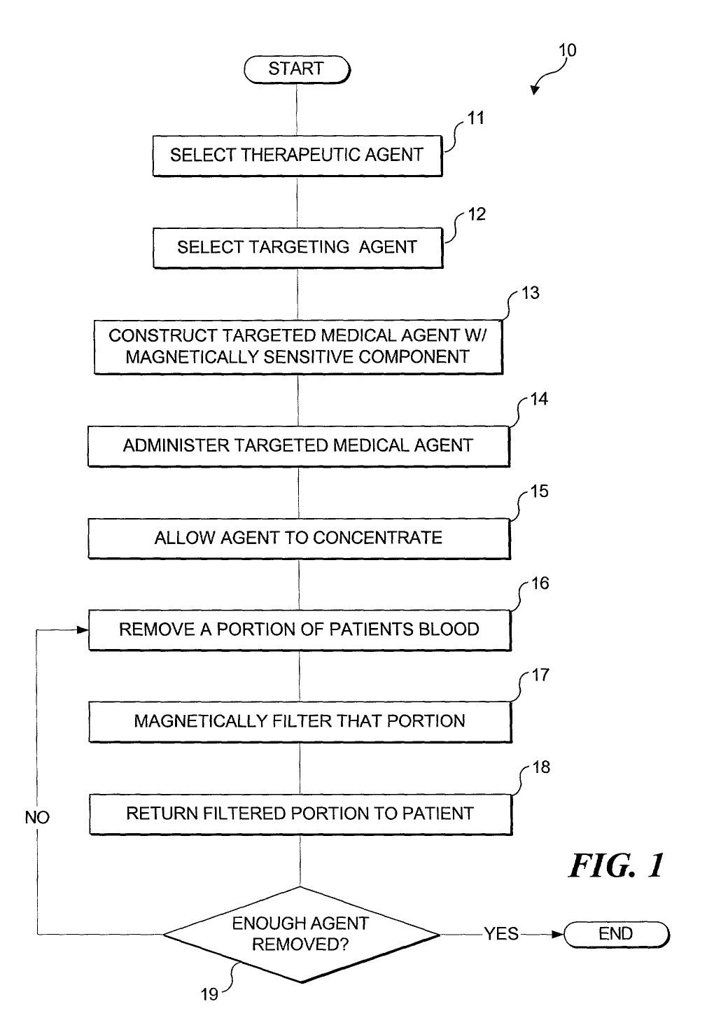

Referring now to the drawings, FIG. 1 shows a flowchart 10 that illustrates the sequence of logical steps employed to treat a patient with a targeted medical agent that includes a magnetically sensitive component, and to reduce an amount of that targeted medical agent that is systemically distributed.

In a block 11, a clinician selects a therapeutic agent to administer to a patient. As noted above, depending on the particular disease condition to be treated, one therapeutic agent will be more appropriate than another will. It should be noted that imaging agents or diagnostic agents (as opposed to therapeutic agents) could also be selected, as appropriate. In a block 12, a specific targeting agent is selected. Preferably, an active targeting agent is employed, such as one of the active targeting agents discussed above. It should be noted that passive targeting agents, such as appropriately sized liposomes as described above, could also be used to produce a targeted medical agent.

Using the selected therapeutic agent (or imaging agent, or diagnostic agent) and the selected targeting agent, the targeted medical agent is produced in a block 13. It should be understood that other components could be incorporated into a targeted medical agent. For example, targeted medical agents are often coated with a polymer, such as PEG, to increase the in vivo residence time of such agents, by making the coated targeted medical agents less likely to be removed by the RES. Carriers, such as microparticles, or encapsulating agents, such as liposomes or micro-bubbles, can also be incorporated into targeted medical agents, as is well known in the art.

Liposomes containing a chemotherapy agent such as doxorubicin may be modified to include a ferromagnetic or superparamagnetic particle, thus making the liposome carriers magnetically sensitive. As a result, the liposome will be retained and separated from a flowing stream of blood by a magnetic field that extends into the flowing stream. Techniques for constructing such “magnetic liposomes” are known in the art. A detailed discussion of magnetic liposomes is presented by [Kubo T, et al. in “Targeted Delivery of Anticancer Drugs With Intravenously Administered Magnetic Liposomes in Osteosarcoma-bearing Hamsters.” Int. J. Oncol. 17: 309-315 (2000).]

The targeting agent selected can be the magnetic particles themselves. For example, if the specific tissue location for treatment is near the dermal layer of the patient, the medical agent may be preferentially localized, or actively targeted to that tissue by placing a permanent magnet or electromagnet in the vicinity of the tissue, thus retaining the medical agent and accelerating the localization of the magnetically targeted medical agents after administration. Specific applications for this targeting paradigm include chemotherapy treatment of skin cancers, head and neck tumors and liver tumors.

In a block 14, the targeted medical agents are administered to the patient. The simplest way to administer the targeted medical agents is in a saline solution that is given with a standard intravenous delivery protocol. A variety of other methods and devices are also suitable for administration of the targeted medical agents into the patient. Two intravascular methods include intra-arterial injection and retrograde venous injection, both of which will improve the localization of carriers in a desired tissue location. Other methods of administration include intramuscular injection and transmucosal and oral delivery. A variety of devices suitable for intravascular administration include standard intravenous administration sets, with the fluid delivered via a pump or by gravity. An implantable, subcutaneous pump of the type that is well known in the art is also a convenient way to deliver the fluid on a continuous or intermittent basis. Intra-arterial and retrograde venous delivery may be accomplished using specialized catheters suitable for these methods. Such devices are well known in the art.

It should be noted that the targeted medical agent could also be administered to the patient in the form of an implanted capsule. Such capsules, which are known in the art, can be produced by binding the therapeutic agents, the targeting agents, and the magnetically sensitive material together with a biodegradable adhesive, forming a “time release” implant that eludes the targeted medical agent in the patient's blood circulation over a sustained period. Polylactic acid polymer is a suitable biocompatible carrier material for such a device, and a variety of adhesives, such as polyvinyl alcohol, may be used as the binder adhesive.

After the targeted medical agent is administered to a patient, a period of time is generally allowed to elapse to enable a portion of the targeted medical agent to accumulate at a target location, as is indicated in a block 15. Note that while some portion of the targeted medical agent does accumulate at the target location, another portion of the targeted medical agent is systemically distributed throughout the body of the patient. The portion of the targeted medical agent systemically distributed in the patient can have undesirable effects on the patient, and is therefore preferably removed by extracorporeally magnetically filtering the patient's bodily fluids in accord with the present invention.

In a block 16, a portion of the patient's bodily fluids (preferably blood, but also lymph or bile) is withdrawn from the patient and passed through a magnetic filter in a block 17 to remove at least a portion of the target medical agent from the withdrawn fluids. For simplicity, the more general term “bodily fluids” is sometimes referred to hereinbelow simply as “blood,” unless from the context it is apparent that the reference is limited only to blood. The magnetically filtered blood is then returned to the patient in a block 18, and the process is repeated (block 19) until the amount of systemically distributed targeted medical agent is reduced. If desired, the blood that is removed can be separated into plasma and non plasma components, and then the plasma can be filtered. However, such a step is not required.

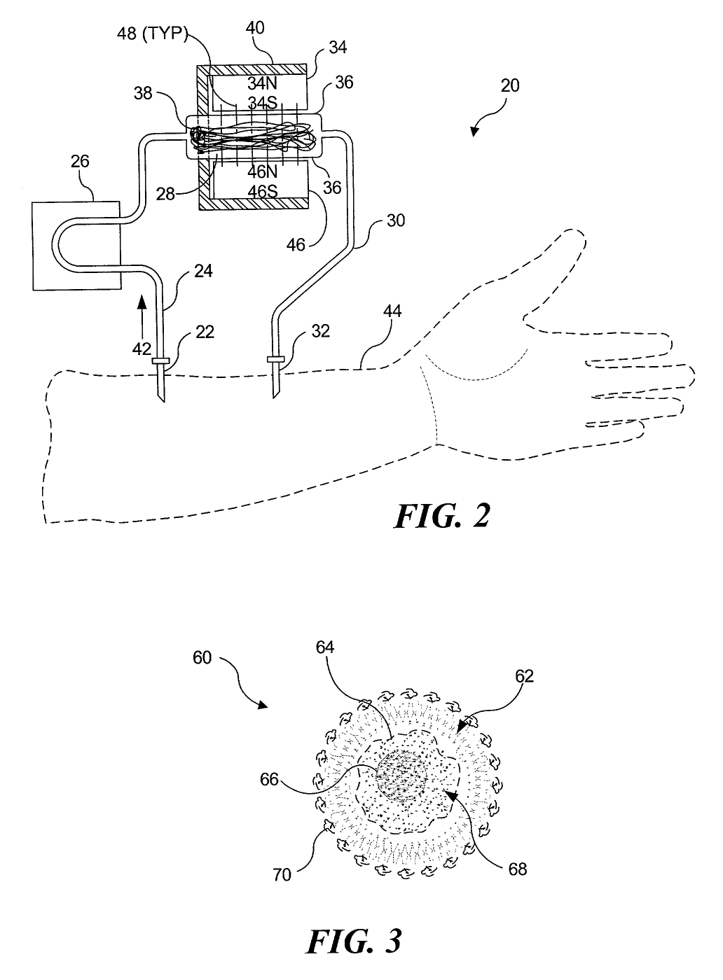

A first embodiment of the extracorporeal magnetic filtering method of the present invention is shown in FIG. 2. An extracorporeal circuit 20 is preferably employed in a continuous process, in which a small portion of a patient's bodily fluids are removed, filtered, and returned in a closed loop process, until a desired systemic reduction of the targeted medical agent is achieved. A pump or arterial pressure can be employed to drive the bodily fluids through the magnetic filter.

Note that extracorporeal circuit 20 removes and returns bodily fluids via different portions of the patient. Extracorporeal circuit 20 includes an inlet needle 32 and an outlet needle 22 disposed in a patient's arm 44. It should be understood that other portions of a patient's body, such as legs, could be used to access bodily fluids. It should also be noted that other suitable bodily fluid access systems are known in the art, such as “single needle” systems commonly used for hemodialysis, and these other systems can also be beneficially employed to remove a patient's bodily fluid for magnetic filtering, in accord with the present invention.

A peristaltic pump 26 draws blood through a tube 24 in a direction 42, and sends the blood through a chamber 28, where the targeted medical agents (which can include a magnetically sensitive component) are removed from the blood. A suitable extracorporeal blood flow rate through the chamber for an average sized adult is about 150-200 milliliters per minute. This flow rate should achieve at least a 90 percent reduction in the amount of systemically distributed targeted medical agents circulating in the blood of the patient in a period of from about 60 minutes to about 90 minutes.

In order to circulate the blood through extracorporeal circuit 20, heparin or another suitable anticoagulant must be added to the blood, which may be accomplished by injecting the heparin directly into the patient via an intravenous injection (not shown) or through a separate metering pump (not shown) that adds the heparin into the blood at any convenient point in tube 24. Heparin management within extracorporeal circuits is a standard hospital practice and is well known in the art.

Chamber 28 has a generally rectangular cross section transverse to the direction of the blood flow and includes two parallel sides 36. Disposed within chamber 28 are a bundle of ferritic stainless steel fibers 38 that concentrate a magnetic flux 48 into the blood flowing through the chamber, where the flux is produced by magnets 34 and 46, which are disposed in close proximity to sides 36. Use of the proper type of stainless steel is important, as both magnetic (ferritic and martensitic) and non-magnetic stainless steels (austenitic) are available. The stainless steel employed must be magnetic to concentrate the magnetic flux within chamber 28. Stainless steel, rather than soft iron, is employed because of the superior anti-corrosion properties of stainless steels, as compared to iron and other iron alloys.

Magnets 34 and 46 are oriented with their poles opposite one another, such that magnetic flux 48 extends from a magnetic south pole 34S to magnetic north pole 46N, passing through chamber 28. A magnetic flux coupler 40, having a high magnetic permeability, further increases the density of magnetic flux 48 between magnetic pole 34S and magnetic pole 46N, by effectively coupling the magnetic flux (not shown) between magnetic poles 34N and 46S. Thus, the amount of magnetic flux not passing through the chamber but instead, extending directly from magnetic pole 34N to magnetic pole 34S, and from magnetic pole 46N to magneticpole 46S is minimized, while the magnetic flux extending through the chamber between magnetic pole 34S and magnetic pole 46N is maximized. Flux coupler 40 is preferably made from a high permeability material, such as iron, mild steel, or the like.

After flowing through chamber 28, the blood flow returns to the patient through a tube 30 that is coupled to inlet needle 32, where it is reintroduced to patient's arm 44. It should be understood that chamber 28 preferably provides a relatively long residence time due to a relatively slow blood velocity through the chamber. Consequently, the blood passing through chamber 28 is exposed to magnetic flux 48 for a sufficiently long time to remove a majority of the targeted medical agents (that incorporate a magnetically sensitive material). A preferred minimum residence time has been empirically determined to be about 5 seconds, and a preferred maximum blood velocity through the magnetic field is about 2 centimeters/second. A preferred magnetic flux density is at least about 0.1 Tesla. A variety of safety features, such as drip chambers, bubble detectors, and pressure monitors (well known in the art), can be incorporated into extracorporeal circuit 20, but have been omitted from FIG. 2 for the sake of clarity.

A simple, alternative method for providing blood flow through extracorporeal circuit 20, without requiring the use of pump 26, is to insert outlet needle 22 into an artery in patient's arm, and to place inlet needle 32 into a vein. In such a configuration, the difference between the patient's arterial and venous blood pressure will cause sufficient blood flow through extracorporeal circuit 20.

FIG. 3 illustrates one embodiment of a passive targeted medical agent 60 that includes a magnetically sensitive component suitable for administration to a patient, such that a quantity of the targeted medical agent is concentrated at a tumor, and a quantity of the targeted medical agent that is systemically distributed throughout the patient's body can be removed in accord with the present invention. Targeted medical agent 60 includes a liposome sphere having a phospholipid bilayer shell 62 and lipid core 64. Within core 64 is a magnetically sensitive particle 66 and a chemotherapy agent 68 (such as doxorubicin). Magnetically sensitive particle 66 can be selected from any of several suitable ferromagnetic, paramagnetic, or superparamagnetic materials such as iron, iron oxide, cobalt, nickel or Heusler alloys (Ni2MnGa). It should be noted that this list is intended to be merely exemplary, rather than limiting the scope of the present invention, since many other materials not listed are also suitable. A coating of PEG molecules 70 are attached to the liposome bilayer to reduce recognition and uptake by the RES of the patient.

For passively targeted medical agents that are targeted to tumors based on their size (i.e., agents that are sized so as to be able to penetrate the capillary walls of the tumor vasculature), preferred sizes range between about 60 and about 400 nanometers in diameter, in order to carry an adequate volume of chemotherapy agent 68, and retaining the ability to penetrate the tumor vasculature.

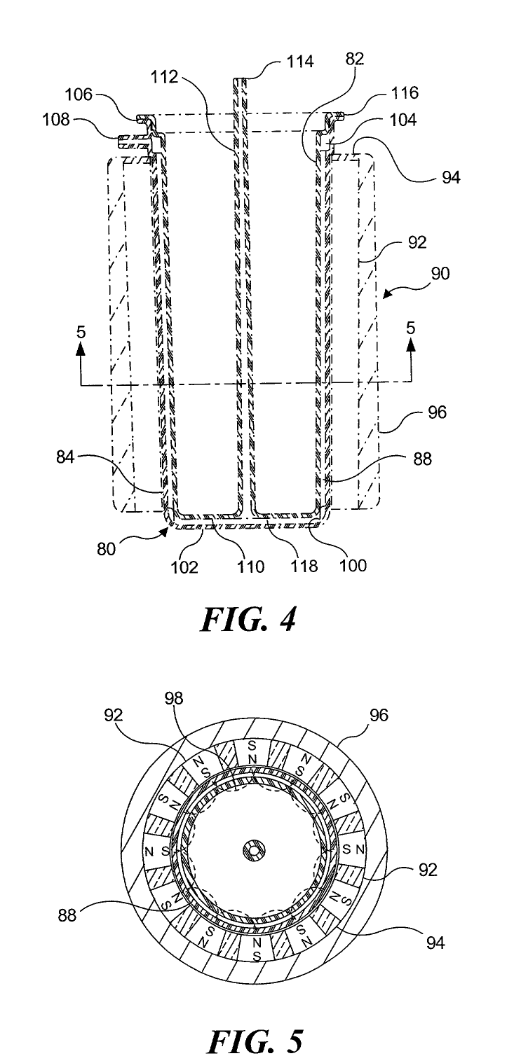

FIGS. 4 and 5 illustrate a magnetic separator chamber 80 and a magnet assembly 90 that can be employed in an extracorporeal circuit to remove agents that include a magnetically sensitive component, in accord with the present invention. For example, magnetic separator chamber 80 and a magnet assembly 90 can take the place of chamber 28 and magnets 34 and 46 in extracorporeal circuit 20 of FIG. 2.

Magnetic separator chamber 80 and magnet assembly 90 are preferably employed to magnetically filter a patient's bodily fluid to remove systemically distributed medical agents that incorporate a magnetically sensitive component, after such a medical agent has been administered to a patient (not shown) by any of the means disclosed above. Note that while medical agents are expected to be therapeutic in nature, other types of medical agents can also be administered for imaging and diagnostic purposes. After administration, and after the desired imaging, diagnostic, or therapeutic procedure has been performed, an extracorporeal circuit (see extracorporeal circuit 20 in FIG. 2) is attached to the patient. Separator chamber 80 is used to remove medical agents that include a magnetically sensitive component from the patient's bodily fluids.

Separator chamber 80 has a cylindrical shape and preferably includes two injection molded parts, an inner housing 82 and outer housing 84. It should be noted that while injection molding represents a preferred fabrication technique, other fabrication techniques could be employed. Outer housing 84 is closed at its bottom by end wall 102 and includes flares at a top end, forming a blood manifold channel 104. Outer housing 84 then flares again to form a lip 106. A fitting 108 is formed in manifold channel 104, to provide an exit for blood flowing through separator chamber 80.

Inner housing 82 is also closed at its bottom by an end wall 110 and includes a fluid path 112 extending along its central axis. Fluid path 112 terminates at a top end of inner housing 82 at a fitting 114 that serves as a blood inlet point, enabling blood to enter into separator chamber 80. Inner housing 82 also flares several times at the top end, providing flares that cooperate with the flares of outer housing 84 to form blood manifold channel 104 and a lip 116 that sealingly engages with lip 106 of outer housing 84. When the inner and outer housing are joined, lip 106 and lip 116 are sealed by any of several bonding methods known in the art, including adhesive bonding, solvent bonding, and ultrasonic welding. Note that an annular volume 88 is defined between inner housing 82 and outer housing 84, when these two housings are joined as described above. As explained below, it is within annular volume 88 that the medical agents incorporating magnetically sensitive components are immobilized and thus filtered from the patient's bodily fluids.

Inner housing 82 and outer housing 84 are joined together to form separator chamber 80. Blood flows into separator chamber 80 via fitting 114, through fluid channel 112, spreading radially into a gap 118 disposed between the bottom portions of inner housing 82 and outer housing 84. The blood (or other bodily fluid) then fills annular volume 88, finally collecting in blood manifold channel 104 and exiting separator chamber 80 through fitting 108. In order to insure a uniform gap between the inner housing 80 and outer housing 84, four centering tabs 100 are molded into the bottom of outer housing 84. The centering tabs ensure a concentric fit between housings 82 and 84. It should be noted that more or fewer centering tabs could be employed, as desired.

Surrounding separator chamber 80 is magnet assembly 90, which preferably includes 12 equally spaced-apart permanent magnets 92, a cage 94 for supporting the permanent magnets, and a flux coupler 96. Preferably, cage 94 is fabricated from a non-magnetic material such as aluminum, polymers, or austentic stainless steel, while coupler 96 is constructed from a material with a high magnetic permeability, such as soft iron. Coupler 96 is preferably an open-ended cylinder that substantially encircles magnets 92. Magnets 92 are arranged with their poles facing inwardly in an alternating north/south pole arrangement. It should be noted that more or fewer magnets could be used, although an even number of magnets is preferred, so that a symmetric alternating pole configuration can be maintained. The arrangement of magnets 92 in conjunction with magnetic flux coupler 96 produces strong magnetic fields 98 between the poles of magnets 92 that extend into annular volume 88, ensuring that any medical agent including a magnetically sensitive material flowing through annular volume 88 is exposed to the magnetic field.

Note that as shown, cylindrical housings 82 and 84 have generally consistent diameters, varying by as little as one or two degrees. Thus, annular volume 88 is substantially uniform in width. However, if it is anticipated that a large volume of magnetically filterable medical agents are to be removed from the bodily fluid, it may be desirable to have a portion of annular volume 88 adjacent the point at which the bodily fluids enter annular volume 88 (i.e., near gap 118) be wider, to prevent an excessive accumulation of magnetically filterable medical agents at that point, which would significantly reduce flow through annular volume 88. The increase in the width of the annular volume at this point is accomplished either by making inner housing 82 narrower at the bottom, or by making outer housing 84 wider at the bottom.

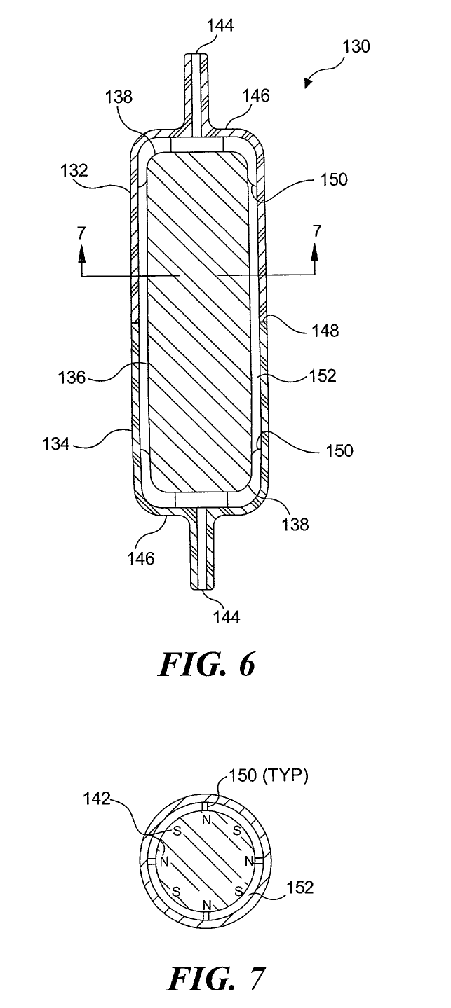

FIGS. 6 and 7 show an alternative embodiment of a magnetic filter 130, in which the magnetic field generator is disposed within the fluid volume. Magnetic filter 130 can readily be incorporated into extracorporeal circuit 20 of FIG. 2 and is formed from only three components, including an upper housing 132, a lower housing 134, and a multipole magnet 136. Preferably magnet 136 is formed as a solid cylinder with radiused ends 138 and includes eight different magnetic poles 142 (see FIG. 6) that extend along its length. Housings 132 and 134 are preferably identical, hollow polymeric cylinders and can readily be molded from a single component. Each housing includes a fitting 144 disposed along a central axis of each housing end wall 146. Upper housing 132 and lower housing 134 are joined together at ajoint 148, using any one of several conventional methods, such as solvent or adhesive bonding.

Preferably disposed on the inside comers of housings 132 and 134 are four centering tabs 150 that help properly position magnet 136, and ensure that a uniform gap exists between each housing and magnet 136. Those of ordinary skill in the art will understand that such tabs could be located in additional, fewer, and/or alternative locations.

Because of the symmetry of housings 132 and 134, either fitting 144 can serve as a fluid inlet, with the opposite fitting 144 serving as a fluid outlet. As fluid containing the magnetically sensitive components (such as the targeted medical agents described above) flows into magnetic filter 130 and into an annular volume 152 formed between the housing and the magnet, medical agents containing magnetically sensitive components are retained by the magnetic field (not shown) extending between magnetic poles 142. A preferred material for magnet 136 is neodymium iron boron, a well known type of magnetic alloy often used to produce a magnet referred to as a rare earth magnet. Such magnets are known for their exceptional magnetic strength compared to conventional iron magnets. Because neodymium magnets are known to corrode readily in an aqueous fluid, magnet 136 is preferably coated with a thin, nonporous, biocompatible coating, such as polyurethane, silicone rubber, polytetrafloroethylene, cross linked polyolefin, or an inert metal plating, such as chromium, gold, or platinum. It should be apparent that magnetic filter 130 could be modified to have a variety of sizes and shapes, and that housings 132 and 134, and magnet 136 in particular could be shaped differently. Furthermore, magnet 136 could be replaced with an array comprising a plurality of individual magnets, as long as annular volume 152 is exposed to a magnetic field having a strength sufficient to immobilize medical agents containing magnetically sensitive components.

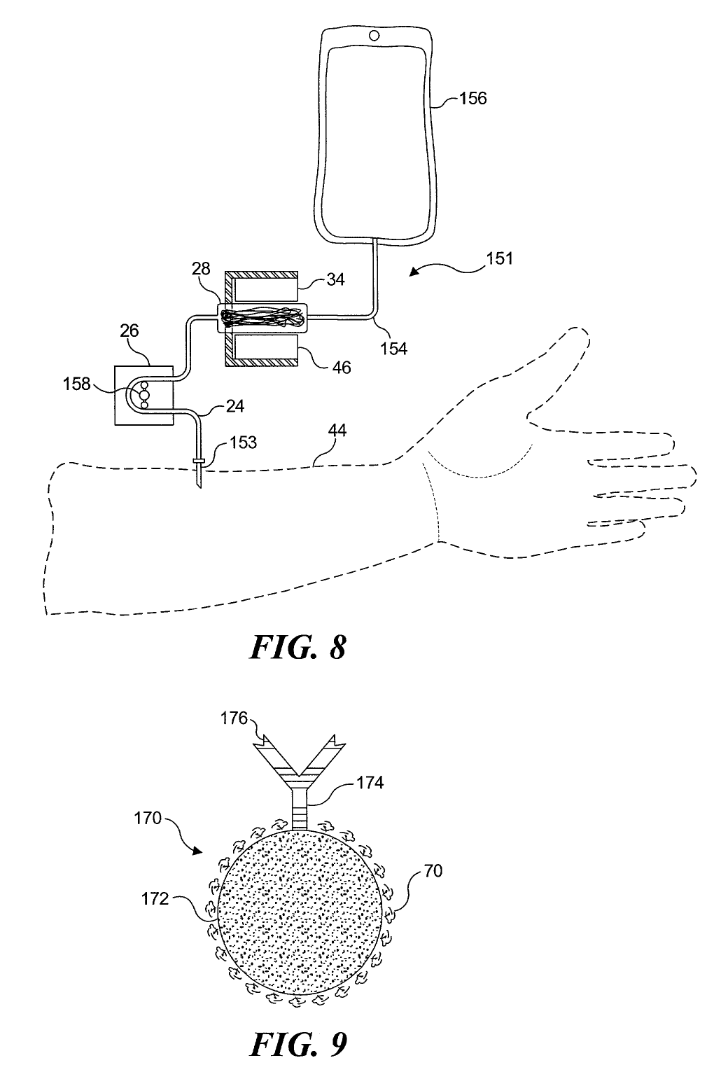

FIG. 8 illustrates an extracorporeal circuit 151 for removing medical agents containing magnetically sensitive components from a patient's bodily fluid using a batch processing method. A single needle 153 is inserted into a vein or artery (not separately shown) in a patient's arm 44. As with extracorporeal circuit 20 of FIG. 2, peristaltic pump 26 draws blood through tube 24 and forces it through chamber 28, where the magnetically sensitive carriers are removed. Note that magnetic filter 130 or magnetic separator chamber 80/magnet assembly 90, each as described previously, could be used in place of chamber 28.

Extracorporeal circuit 151 differs from extracorporeal circuit 20 in that instead of returning a bodily fluid directly to a patient, the blood (or other bodily fluid) travels through a tube 154 into a collection reservoir 156, where it is treated as a batch. Reservoir 156 may be the closed flexible container as shown, or a rigid container with a sterile vent to the atmosphere. Such containers are well known in the medical art. Preferably, pump 26 is a peristaltic roller pump having a control system (not shown) that monitors the amount of blood removed from the patient, e.g., by counting the total number of revolutions of a pump head 158. By utilizing tube 24 having a known size, a specific volume of blood may be pumped from the patient and into reservoir 156, simply by controlling the number of pump cycles applied. Once a desired quantity of blood has been removed and filtered, the pump is reversed, and the filtered blood is pumped back into the patient through tube 24 and needle 153.

Extracorporeal circuit 151 processes blood in a batch mode and advantageously requires only one access needle into the patient's vasculature. In the batch processing mode, the blood flow rate should be preferably increased to about 200-400 milliliters per minute in order to achieve the desired 90 percent reduction of medical agents with magnetically sensitive components within a period of about 60 minutes to about 90 minutes.

FIG. 9 illustrates a targeted medical agent 170 that includes an antibody targeting component and a magnetically sensitive component. During the treatment of a patient, a portion of targeted medical agent 170 is accumulated in a target area, and a portion of targeted medical agent 170 is systemically distributed throughout a patient's body. The systemically distributed portion is then removed with a magnetic filter in accord with the present invention.

The therapeutic action of targeted medical agent 170, for example, can employ radiation therapy for treatment of lymphoma. In this application, a core 172 comprises a mixture of a polymer substrate (such as albumin), a magnetically sensitive material (such as iron oxide), and a therapeutic radionuclide (such as iodine-131). In order to increase the circulating time of targeted medical agent 170 in a patient's blood, PEG molecules 70 are attached to the surface of targeted medical agent 170. To provide a targeting function, a targeting antibody 174 is also attached to the surface of targeted medical agent 170. For example, monoclonal antibody Anti B-1 can be used to target and bind to CD-20 positive cells expressed by malignant B lymphocytes using receptor sites 176 located on the ends of the antibody.

A method of treating the patient with targeted medical agent 170 includes the following steps. First, a patient is given a small, intravenous injection of antibody Anti B-1 only, in order to determine if an allergic reaction will occur. Next, a whole body gamma scan is performed to provide a baseline measurement. Then, a suspension of targeted medical agent 170 in a normal saline solution is administered intravenously. A few hours after the medical agent is administered, a second gamma scan is performed in order to assess the level of attachment of targeted medical agent 170 to the lymphocytes. If adequate, an extracorporeal circuit such as those illustrated in FIG. 2 or 8 is attached to the patient, and the remaining, systemically circulating portion of targeted medical agents 170 is removed by magnetically filtering the patient's bodily fluids, as explained above.

Although the present invention has been described in connection with the preferred form of practicing it, those of ordinary skill in the art will understand that many modifications can be made thereto within the scope of the claims that follow. Accordingly, it is not intended that the scope of the invention in any way be limited by the above description, but instead be determined entirely by reference to the claims that follow.

The invention in which an exclusive right is claimed is defined by the following:

1. A method for administering a medical agent to a patient, and then substantially removing excess medical agent from the patient, comprising the steps of:
(a) selecting a medical agent that is capable of inducing a desired effect in the patient;
(b) adding a magnetically sensitive material to the medical agent, producing a magnetically sensitive medical agent;
(c) administering the magnetically sensitive medical agent to the patient;
(d) enabling the magnetically sensitive medical agent to induce the desired effect in the patient; and
(e) removing the unused magnetically sensitive medical agent from the patient by:
(i) removing a portion of a bodily fluid from the patient, said bodily fluid carrying the unused magnetically sensitive medical agent;
(ii) magnetically filtering said portion of the bodily fluid to remove the magnetically sensitive medical agent, producing filtered bodily fluid;
(iii) returning the filtered bodily fluid to the patient; and
(iv) repeating steps (i)-(iii) until the unused magnetically sensitive medical agent has been substantially removed from the patient.

2. The method of claim 1, wherein the step of enabling the magnetically sensitive medical agent to induce the desired effect in the patient comprising the step of concentrating the magnetically sensitive medical agent at a target location by allowing sufficient time to elapse for the magnetically sensitive medical agent to concentrate at the target location.

3. The method of claim 1, wherein the step of administering the magnetically sensitive medical agent to the patient comprises the step of administering the magnetically sensitive medical agent by at least one of an intra-arterial injection and a retrograde venous injection.

4. The method of claim 1, wherein the step of administering the magnetically sensitive medical agent to the patient comprises the step of administering the magnetically sensitive medical agent by at least one of implantation and orally.

5. The method of claim 1, wherein the bodily fluid comprises blood, and the step of removing the portion of the bodily fluid, comprises the step of adding an anticoagulant to the blood.

6. The method of claim 1, wherein the bodily fluid comprises at least one of a lymph fluid and a bile fluid.

7. The method of claim 1, wherein the step of removing bodily fluid is executed as a substantially continuous process and includes the step of circulating the portion of the bodily fluid through an extracorporeal circuit.

8. The method of claim 7, wherein the step of circulating the portion of the bodily fluid includes the step of pumping the bodily fluid.

9. The method of claim 7, wherein the step of circulating a portion of the bodily fluid is facilitated using an arterial pressure.

10. The method of claim 1, wherein the step of removing bodily fluid is executed as a batch process.

11. The method of claim 10, wherein the batch process comprises the steps of:
(a) pumping a desired batch of the bodily fluid from the patient through a magnetic field;
(b) collecting the bodily fluid removed from the patient;
(c) magnetically filtering the batch of the bodily fluid to remove the magnetically sensitive medical agent, producing the filtered bodily fluid; and
(d) pumping the filtered bodily fluid back into the patient.

12. The method of claim 1, further comprising the step of adding a polymer surface coating to the medical agent to increase an in vivo residence time of the medical agent.

13. The method of claim 1, wherein when the medical agent comprises a radioactive material, further comprising the step of performing a whole body scan to determine a baseline measure of radioactivity within the patient, before the step of administering the magnetically sensitive medical agent.

14. A method for administering a targeted medical agent to a patient, such that the targeted medical agent is concentrated at a targeted location, and unused targeted medical agent is substantially removed from non-targeted locations within the patient, comprising the steps of:
(a) selecting a medical agent that is capable of inducing a desired effect at a targeted location in the patient;
(b) combining said medical agent with a targeting component, producing a targeted medical agent;
(c) adding a magnetically sensitive material to said combination, producing a magnetically sensitive targeted medical agent;
(d) administering the magnetically sensitive targeted medical agent to the patient;
(e) enabling the magnetically sensitive targeted medical agent to reach the targeted location; and
(f) removing the magnetically sensitive targeted medical agent from non-targeted portions within the patient by:
(i) removing a portion of a bodily fluid from the patient, said bodily fluid carrying the unused magnetically sensitive target medical agent through the non-targeted locations in the patient;
(ii) magnetically filtering said portion of the bodily fluid to remove the magnetically sensitive targeted medical agent, producing filtered bodily fluid;
(iii) returning the filtered bodily fluid to the patient; and
(iv) repeating steps (i)-(iii) until the unused magnetically sensitive targeted medical agent has been substantially removed from the non-targeted locations in the patient.

15. The method of claim 14, further comprising the step of concentrating the magnetically sensitive targeted medical agent at the targeted location by allowing sufficient time to elapse for the magnetically sensitive targeted medical agent to concentrate at the targeted location.

16. The method of claim 14, wherein the step of concentrating comprises the step of waiting at least four hours before initiating the step of removing the magnetically sensitive targeted medial agent from the non-targeted portions within the patient.

17. The method of claim 14, wherein the step of administering the magnetically sensitive targeted medical agent to the patient comprises the step of administering the magnetically sensitive targeted medical agent by intravascular injection.

18. The method of claim 17, wherein the step of administering the magnetically sensitive targeted medical agent by intravascular injection comprises the step of employing at least one of an intra-arterial injection and a retrograde venous injection.

19. The method of claim 14, wherein the step of administering the magnetically sensitive targeted medical agent to the patient comprises the step of administering the magnetically sensitive targeted medical agent by implantation.

20. The method of claim 14, wherein the step of administering the magnetically sensitive targeted medical agent to the patient comprises the step of administering the magnetically sensitive targeted medical agent orally.

21. The method of claim 14, wherein the bodily fluid comprises blood.

22. The method of claim 21, wherein the step of removing the portion of the bodily fluid, comprises the step of adding an anticoagulant to the blood.

23. The method of claim 14, wherein the bodily fluid comprises at least one of a lymph fluid and a bile fluid.

24. The method of claim 14, wherein the step of removing bodily fluid is executed as a substantially continuous process and includes the step of circulating the portion of the bodily fluid through an extracorporeal circuit.

25. The method of claim 24, wherein the step of circulating the portion of the bodily fluid includes the step of pumping the bodily fluid.

26. The method of claim 24, wherein the step of circulating a portion of the bodily fluid is facilitated using an arterial pressure.

27. The method of claim 24, wherein the step of circulating a portion of the bodily fluid comprises the step of employing a flow rate of from about 150 to about 200 milliliters per minute.

28. The method of claim 14, wherein the step of magnetically filtering the bodily fluid comprises the step of exposing the bodily fluid to a magnetic field for at least about five seconds.

29. The method of claim 14, wherein the step of magnetically filtering the bodily fluid comprises the step of exposing the bodily fluid to a magnetic field such that a velocity of the bodily fluid through the magnetic field is less than about two centimeters per second.

30. The method of claim 14, wherein the step of magnetically filtering the bodily fluid comprises the step of exposing the bodily fluid to a magnetic field such that a flow rate of the bodily fluid through the magnetic field is less than about 200 milliliters per minute.

31. The method of claim 14, wherein the step of magnetically filtering the bodily fluid comprises the step of exposing the bodily fluid to a magnetic field such that a flow rate of the bodily fluid through the magnetic field is less than about 150 milliliters per minute.

32. The method of claim 14, wherein the step of magnetically filtering the bodily fluid comprises the step of exposing the bodily fluid to a magnetic field having an intensity greater than about 0.1 Tesla.

33. The method of claim 14, wherein the step of removing bodily fluid is executed as a batch process.

34. The method of claim 33, wherein the batch process comprises the steps of:
(a) pumping a desired batch of the bodily fluid from the patient through a magnetic field;
(b) collecting the bodily fluid removed from the patient;
(c) magnetically filtering the batch of the bodily fluid to remove the magnetically sensitive targeted medical agent, producing the filtered bodily fluid; and
(d) pumping the filtered bodily fluid back into the patient.

35. The method of claim 14, further comprising the step of adding a polymer surface coating to the medical agent to increase an in vivo residence time of the medical agent.

36. The method of claim 14, wherein the step of combining comprises the step of limiting a size of the targeted medical agent to between about 60 and about 400 nanometers.

37. The method of claim 14, further comprising the step of encapsulating the selected medical agent in a shell that is about 100 nanometers in size before the step of combining the medical agent with a targeting component.

38. The method of claim 14, wherein when the targeting component comprises an antibody, further comprising the step of administering a test amount of the antibody alone to the patient, to determine if an allergic reaction occurs, before the step of administering the magnetically sensitive targeted medical agent.

39. The method of claim 14, wherein when the medical agent comprises a radioactive material, further comprising the step of performing a whole body scan to determine a baseline measure of radioactivity within the patient, before the step of administering the magnetically sensitive targeted medical agent.

40. The method of claim 14, wherein when the medical agent comprises a radioactive material, further comprising the step of performing a whole body scan to determine if sufficient radioactive material is concentrated at the target location to achieve a desired effect, before the step of removing the unused targeted medical agent.

41. The method of claim 14, wherein the step of concentrating the magnetically sensitive targeted medical agent at the targeted location does not employ a magnetic field to concentrate the magnetically sensitive targeted medical agent at the targeted location.

42. A method for administering a medical agent to a patient, such that the medical agent is concentrated at a targeted location, but is substantially removed from non-targeted locations within the patient, comprising the steps of:
(a) selecting a medical agent that is capable of inducing a desired effect at the targeted location in a patient;
(b) combining said medical agent with a targeting component that preferentially is attracted to tissue at the targeted location, producing a targeted medical agent;
(c) adding a magnetically sensitive material to said targeted medical agent, producing a magnetically sensitive targeted medical agent;
(d) administering the magnetically sensitive targeted medical agent to apatient;
(e) concentrating the magnetically sensitive targeted medical agent at the targeted location; and
(f) removing the magnetically sensitive targeted medical agent from non-targeted portions of the patient to which it is systemically distributed, by:
(i) removing a portion of a bodily fluid from the patient, said bodily fluid conveying the magnetically sensitive targeted medical agent systemically within the patient;
(ii) magnetically filtering said portion of the bodily fluid to remove the magnetically sensitive targeted medical agent from said portion of the bodily fluid, producing filtered bodily fluid;
(iii) returning the filtered bodily fluid to the patient; and
(iv) repeating steps (i)-(iii) until the bodily fluid has been sufficiently filtered to substantially remove the magnetically sensitive targeted medical agent being systemically distributed within the patient.

43. A method for administering a medical agent to a patient, such that the medical agent is concentrated at a targeted location, but is substantially removed from non-targeted locations within the patient, comprising the steps of:
(a) selecting a medical agent that is capable of inducing a desired effect at a targeted location in the patient;
(b) producing a magnetically sensitive medical agent by combining said medical agent with a magnetically sensitive material;
(c) administering the magnetically sensitive medical agent to the patient;
(d) concentrating the magnetically sensitive medical agent at the targeted location, by providing a magnetic field adjacent to the targeted location, said magnetic field attracting the magnetically sensitive material of the magnetically sensitive medical agent to the targeted location; and
(e) removing the magnetically sensitive medical agent from non-targeted locations of the patient by:
(i) withdrawing a portion of a bodily fluid from the patient, said bodily fluid including the magnetically sensitive medical agent;
(ii) magnetically filtering said portion of the bodily fluid that has been withdrawn, to remove the magnetically sensitive medical agent from said portion of the bodily fluid producing filtered bodily fluid;
(iii) returning the filtered bodily fluid to the patient; and
(iv) repeating steps (i)-(iii) until the bodily fluid has been sufficiently filtered to substantially remove the magnetically sensitive medical agent being systemically distributed within the patient.

44. The method of claim 43, further comprising the step of retaining at least a portion of said magnetically sensitive medical agent at the targeted location, by maintaining a magnetic field adjacent to the targeted location.

45. The method of claim 44, wherein the magnetic field is maintained adjacent to the targeted location for as long as it is desirable for the magnetically sensitive medical agent to be retained at the target location.

46. The method of claim 43, wherein the step of producing a magnetically sensitive medical agent comprises the step of by encapsulating said medical agent and said magnetically sensitive material within one of a liposome and a polymer shell.

47. The method of claim 43, further comprising the step of coating said medical agent with a polymer that reduces an uptake of the medical agent by a reticuloendothelial system of the patient.

48. The method of claim 43, wherein the step of withdrawing the portion of the bodily fluid comprises the step of removing the bodily fluid from a first location on the patient through a fluid line; wherein the step of magnetically filtering comprises the steps of pumping the bodily fluid that was removed into a collection reservoir, and exposing the bodily fluid to a magnetic filter; and wherein the step of returning the filtered bodily fluid to the patient comprises the step of pumping the filtered bodily fluid back through the fluid line and into the patient.

49. The method of claim 48, wherein the step of withdrawing further comprises the step of controlling an amount of bodily fluid removed from the patient, so that only a desired amount is removed.

50. The method of claim 43, wherein the step of withdrawing comprises the step of removing the portion of the bodily fluid from a first location on the patient through a first fluid line; and wherein the step of returning the filtered bodily fluid to the patient comprises the step of returning the filtered bodily fluid to a second location on the patient through a second fluid line.

51. The method of claim 50, further comprising the step of employing a pump to drive the portion of the bodily fluid withdrawn and the filtered bodily fluid through the first and second fluid lines, respectively.

52. The method of claim 50, further comprising the step of employing arterial pressure to drive the portion of the bodily fluid withdrawn and the filtered bodily fluid through the first and second fluid lines, respectively.

53. A method for reducing an amount of targeted medical agent systemically distributed in a patient, while enabling said targeted medical agent to be concentrated at a target location in the patient, comprising the steps of:
(a) administering targeted medical agent to the patient, said targeted medical agent comprising a medical agent, a magnetically sensitive component, and a targeting component; and
(b) extracorporeally magnetically filtering a bodily fluid withdrawn from the patient to reduce an amount of the targeted medical agent systemically distributed in the patient.

54. A method for reducing an amount of targeted medical agent systemically distributed in a patient, while enabling said targeted medical agent to concentrate at a target location in the patient, comprising the steps of:
(a) administering targeted medical agent to the patient, said targeted medical agent comprising a medical agent, a magnetically sensitive component, and a targeting component;
(b) allowing sufficient time to elapse for at least a portion of said targeted medical agent to become concentrated at the target location; and
(c) extracorporeally magnetically filtering a bodily fluid withdrawn from the patient to reduce an amount of targeted medical agent systemically distributed in the patient.

55. A system for reducing a systemic concentration of a medical agent in a patient, comprising:
(a) means for removing a bodily fluid from a patient, wherein said bodily fluid contains a medical agent that includes a magnetically attracted component;
(b) a fluid volume having an inlet and an outlet, said inlet and outlet being adapted to couple in fluid communication with a patient and to convey a bodily fluid from and to a patient; and
(c) a magnetic field generator disposed adjacent the fluid volume and thereby adapted to act on a bodily fluid removed from a patient that is contained within the fluid volume, said magnetic field generator producing a magnetic field adapted to attract a magnetically attracted component contained in a bodily fluid, so that a medical agent is filtered from a bodily fluid.

56. A system for reducing a systemic concentration of a medical agent in a patient, comprising:
(a) a fluid line adapted to couple in fluid communication with a patient, to enable a bodily fluid to be removed from a patient through said fluid line;
(b) means for magnetically filtering medical agent from a bodily fluid that has been removed from a patient, wherein the medical agent includes a component attracted to a magnetic field, said means for magnetically filtering comprising:
(i) a housing defining a fluid volume and having an inlet and an outlet, said inlet being coupled in fluid communication with said fluid line; and
(ii) a magnetic field generator disposed proximate to said fluid volume, producing a magnetic field that extends into said fluid volume; and
(c) a filtered fluid line in fluid communication with said outlet, said filtered fluid line being adapted to couple in fluid communication with a patent to return a filtered bodily fluid to a patient.

57. A system for administering a medical agent to a patient, such that a first portion of said medical agent is concentrated at a target location, and a second portion of said medical agent that is not disposed at the target location is substantially removed from the patient; comprising:
(a) a targeted medical agent comprising a therapeutic component, a targeting component that causes said targeted medical agent to be concentrated at a target location within a patient, and a magnetically sensitive component;
(b) means for administering said targeted medical agent to a patient;
(c) means for removing a bodily fluid from a patient, wherein said bodily fluid contains at least a portion of the targeted medical agent administered to a patient;
(d) a fluid line adapted to couple a patient in fluid communication with said means for removing a bodily fluid from a patient;
(e) means for magnetically filtering targeted medical agent from a bodily fluid withdrawn from a patient, said means for magnetically filtering comprising
(i) a fluid volume having an inlet and an outlet, said inlet being in fluid communication with said fluid line; and
(ii) a magnetic field generator disposed proximate said fluid volume and producing a magnetic field extending into said fluid volume, a strength of said magnetic field being sufficient to immobilize substantially all targeted medical agent within said fluid volume; and
(f) a filtered fluid line adapted to couple a patient in fluid communication with said outlet, to convey a magnetically filtered bodily fluid from which said targeted medical agent has been substantially removed, back into a patient.

58. The system of claim 57, wherein said targeted medical agent further comprises at least one of a liposome, a protein, a lipid, a polymer, a peptide, a lipopolymer, a gas bubble, a biological cell, a virus, a bacteria, a prion, an antibody, an antigen, a hydrogel, and a dendrimer.

59. The system of claim 57, wherein said means for administering said targeted medical agent to a patient comprises one of a syringe, a pump, an administration set utilizing gravity flow, and an implanted device.

60. A system for reducing a systemic concentration of a medical agent incorporating a magnetically sensitive component in a patient, comprising:
(a) a first fluid line adapted to couple in fluid communication with a patient, such that a bodily fluid can be removed from a patient through said first fluid line;
(b) means for magnetically filtering the medical agent from a bodily fluid, said means for magnetically filtering comprising:
(i) a fluid volume having an inlet and an outlet, said inlet being in fluid communication with said first fluid line; and
(ii) a magnetic field generator disposed proximate to said fluid volume, producing a magnetic field substantially extending into said fluid volume, a strength of said magnetic field being sufficient to immobilize substantially all medical agent conveyed by a bodily fluid into said fluid volume, producing a filtered bodily fluid;
(c) a second fluid line in fluid communication with said outlet;
(d) a fluid reservoir for temporary storage of the filtered bodily fluid; and
(e) a pump operatively coupled to one of said first and second fluid lines, said pump withdrawing a bodily fluid from a patient through said fluid volume and into said fluid reservoir, and drawing filtered bodily fluid from said fluid reservoir for return to a patient.

61. A system for reducing a systemic concentration of a medical agent in a patient, said medical agent being attracted to a magnetic field, comprising:
(a) means for removing a bodily fluid from a patient, wherein said bodily fluid contains at least a portion of a medical agent in a patient;
(b) a magnetic filter for magnetically filtering the medical agent from a bodily fluid that has been withdrawn from a patient, said magnetic filter comprising:
(i) a magnetic separator chamber comprising a generally annular volume defined between a generally cylindrical inner component and a generally cylindrical outer component, said generally annular volume having a fluid inlet and a fluid outlet, said fluid inlet and said fluid outlet being generally disposed at opposing ends of said generally annular volume and in fluid communication with said means for removing a bodily fluid; and
(ii) a magnetic field generator disposed adjacent to said generally annular volume and producing a magnetic field having an intensity of about at least 0.1 Tesla within said annular volume, said magnetic field filtering the medical agent from the bodily fluid, producing a magnetically filtered bodily fluid; and
(c) means for returning a magnetically filtered bodily fluid to a patient, wherein such a magnetically filtered bodily fluid contains substantially less medical agent than was present in an unfiltered bodily fluid removed from a patient.

62. The system of claim 61, wherein said magnetic field generator comprises a plurality of elongate magnets disposed in a spaced-apart array about an outer surface of said generally cylindrical outer component, adjacent magnets having a different pole oriented towards said generally annular volume, such that said generally annular volume is exposed to a magnetic field extending between the different poles.

63. The system of claim 62, wherein said magnetic field generator further comprises a flux coupler that enhances a magnetic flux directed toward said generally annular volume.

64. The system of claim 61, wherein said fluid inlet and fluid outlet are disposed generally at opposite ends of said magnetic filter.

65. The system of claim 61, wherein a diameter of at least one of said generally cylindrical inner component and said generally cylindrical outer component varies along its longitudinal axis, such that a spacing between said generally cylindrical inner component and said generally cylindrical outer component is larger adjacent said inlet than adjacent to said outlet, so that particles comprising the medical agent that immobilized by said magnetic filter do not accumulate within the annular volume sufficiently to block said inlet.

66. A magnetic filter capable of immobilizing a magnetically attracted material entrained in a fluid, comprising:
(a) a magnetic separator chamber comprising a generally annular volume defined between a generally cylindrical inner component and a generally cylindrical outer component, said generally annular volume comprising a fluid inlet and a fluid outlet, said fluid inlet and said fluid outlet being generally disposed at opposite ends of said magnetic separator chamber; and
(b) a magnetic field generator disposed adjacent to said magnetic separator chamber, such that said generally annular volume is exposed to a magnetic field produced by the magnetic field generator, immobilizing any magnetically attracted material entrained in a fluid flowing through the annular volume.

67. The magnetic filter of claim 66, wherein said magnetic field generator is capable of producing a magnetic field of at least about 0.1 Tesla within said generally annular volume.

68. The magnetic filter of claim 66, wherein said magnetic field generator is disposed adjacent an outer surface of said generally cylindrical outer component.

69. The system of claim 66, wherein said magnetic field generator comprises a plurality of elongate magnets disposed in a spaced-apart array adjacent to said magnetic separator chamber, adjacent magnets having a different pole oriented towards said generally annular volume.

70. The system of claim 69, wherein said magnetic field generator further comprises a flux coupler that enhances a magnetic flux directed toward said generally annular volume.

71. The system of claim 66, wherein said magnetic field generator exposes said generally annular volume to a magnetic field.

72. The system of claim 66, wherein at least one of said fluid inlet and fluid outlet comprises a fluid channel disposed along a central axis of said generally cylindrical inner component.

73. The system of claim 66, wherein a diameter of at least one of said generally cylindrical inner component and said generally cylindrical outer component varies along its longitudinal axis, such that a spacing between said generally cylindrical inner component and said generally cylindrical outer component is larger adjacent said fluid inlet than adjacent to said fluid outlet, so that the magnetically attracted material that is immobilized by said magnetic field does not accumulate sufficiently to block said inlet.
